(19) United States
(12) Patent Application Publication
(10) Pub. No.: US 2007/0099947 A1
(43) Pub. Date: May 3, 2007

(54) Methods and compositions for the treatment of brain reward system disorders by combination therapy

(75) Inventors: Reginald L. Dean III, Boxborough, MA (US); Daniel Deaver, Franklin, MA (US); Elliot Ehrich, Lincoln, MA (US)

Correspondence Address: ELMORE PATENT LAW GROUP, PC, 209 MAIN STREET, N. CHELMSFORD, MA, 01863, US

(73) Assignee: Alkermes, Inc., Cambridge, MA (US)

(21) Appl. No.: 11/591,842

(22) Filed: Nov. 2, 2006

Related U.S. Application Data
(60) Provisional application No. 60/733,050, filed on Nov. 3, 2005.

(51) Int. Cl. A61K 31/485; A61K 31/4745

(52) U.S. Cl. 514/282; 514/295

(57) ABSTRACT

The present invention is directed to a combination treatment of an opioid antagonist e.g., naltrexone and a second compound selected from the group consisting of a GABA B agonist, an NMDA antagonist, a serotonin antagonist, and a cannabinoid antagonist is the key to the successful treatment of a brain reward system disorder. A brain reward system, include but are not limited, to pathological gambling, compulsive alcohol consumption, compulsive over-eating and obesity, compulsive smoking, and drug addiction. The compounds and methods of the present invention effectively reduce the cravings, withdrawal symptoms and negative drug side effects associated with a monotherapy. As such, patient compliance is greatly increased, thereby decreasing relapse of a brain reward system disorder.

### RELATED APPLICATION

This application claims the benefit of U.S. Provisional Application No. 60/733,050, filed on Nov. 3, 2005. The entire teaching of the above application is incorporated herein by reference.

### TECHNICAL FIELD

The present invention relates to a combination therapy for the treatment of disorders associated with the brain reward system.

### BACKGROUND OF THE INVENTION

The brain's reward system serves to reinforce healthy behavior. Dopamine, a neurotransmitter associated with pleasant or euphoric feelings, is released by these reward areas to encourage the body to repeat these healthy behaviors. However, drugs like nicotine, heroine or cocaine that stimulate the brain can activate these normal reinforcement pathways, providing the same rewards for harmful behaviors. Compulsive or excessive behaviors also have many affinities to addictive behavior e.g., substance abusers. For example, compulsive behavior such as gambling can produce the same aroused euphoria as those experienced by addicts e.g., substance abusers. For example, pathological gamblers express a distinct craving for the “feel” of gambling; they develop tolerance in that they need to take progressively greater risks and make progressively larger bets to reach a desired level of excitement. They crave the unhealthy stimulus and experience withdrawal-like symptoms when no “action” is available.

The brain reward system is a specialized circuitry of the brain involving the mesocorticolimbic dopaminergic system. The dopaminergic system is activated by healthy behaviors such as food consumption, sexual activity and parental care. Dopaminergic activation enhances the occurrence of these healthy behaviors. However, the same feelings of gratification often experienced from these healthy activities have been implicated in addictive behavior e.g., substance abusers. Researchers further suggest a link between dopaminergic neurotransmission and a range of compulsive or excessive behaviors e.g., gambling, over-eating, or kleptomania. Although many complex factors may be involved in compulsive or excessive behaviors, the main similarity is that the behavior causes the brain to change, reward circuits are disrupted, and the compulsive or excessive behavior eventually becomes involuntary.

Treatments regimes available for addictive behavior e.g., substance abusers, include medication, detoxification and rehabilitation. In contrast, psychotherapy is the main treatment available for individuals afflicted from compulsive or excessive behaviors. However, these treatments often do not address the full spectrum of negative aspects associated with abstinence from the addictive or excessive or compulsive behavior. For example, medications such as diazepam or methadone, used to wean a substance abuser from the addictive behavior often cause addiction to the treatment medication itself. Furthermore, substance abusers and individuals afflicted with an excessive or compulsive behavior experience cravings and withdrawal or withdrawal-like symptoms in the absence of the harmful stimulus. In addition, patients also experience adverse clinical manifestations to the treatment medication itself, for example, negative drug side effects e.g., nausea. These cravings, withdrawal symptoms and negative drug side effects often lead to a lack of patient compliance and relapse.

Interest in the use of opioid antagonists for treating addiction beyond opiates arose from theories that the endogenous opioid system mediates many of the reinforcing attributes of the addiction through the release of dopamine (e.g., animal and human studies in support of this involvement with alcohol are reviewed in O'Leary, et al., 2001; Oswald and Wand, 2004). Various studies have since examined the potential therapeutic effects of naltrexone in a number of different addictive or compulsive disorders (reviewed in Modesto-Lowe and Van Kirk, 2002). Additionally, drugs known to modulate, dampen or reduce dopamine levels in brain areas associated with reward have also been evaluated as treatment options for alcohol dependency (Mann, 2004), substance abuse (Vetulani, 2001; Gentry, et al., 2002; Cornish, et al., 2004), pathological gambling (Kim, et al., 2002), eating disorders (Agras, 2004; Gold and Star, 2005), and nicotine/tobacco addiction (Henningfield, et al., 2005).

As such, a further need exists for effective treatments to treat the full spectrum of negative aspects associated with addiction and excessive or compulsive behaviors. In particular, there is a need for effective treatments against the cravings, withdrawal symptoms and negative drug side effects associated with abstinence from a brain reward system disorders.

### SUMMARY OF THE INVENTION

The present invention is directed to a combination treatment of an opioid antagonist, e.g., naltrexone and its analogs and derivatives, and a second compound selected from the group consisting of a GABA B agonist, an NMDA antagonist, a serotonin antagonist, and a cannabinoid antagonist for the successful treatment of a disorder associated with the brain reward system. Brain reward system disorders are characterized by an inability to refrain from repeatedly engaging in an addictive behavior e.g., nicotine/tobacco, alcohol and/or drug abuse, or compulsive or excessive behaviors e.g., pathological gambling and/or compulsive over-eating and obesity. Individuals who abstain from an addictive or excessive or compulsive behavior often experience cravings and withdrawal symptoms. The combination treatment produces a synergistic or additive effect on a disorder associated with the brain reward system. For example, the combined effect of administering two therapeutic compounds produces an overall response that is greater than the sum of the two individual effects. Furthermore, the synergistic or additive effect of the combined therapy allows for a lower dosing regime than that currently available in the market place for a monotherapy. In turn, the compounds and methods of the present invention effectively reduce the cravings, withdrawal symptoms and negative drug side effects associated with a monotherapy. As such, patient compliance is greatly increased, thereby decreasing relapse of a brain reward system disorder.

The current invention provides a composition for the treatment of brain reward system disorders comprising concurrently administering to a subject in need of treatment a therapeutically effective amount of: (i) a first compound comprising an opioid antagonist or a pharmaceutically acceptable salt, isomer, prodrug, analog, metabolite or derivative thereof; and (ii) and a second compound effective to ameliorate or eliminate at least one symptom of brain reward system disorders; wherein the combined therapy potentiates the therapeutic response compared to treatment of either compound as monotherapy.

In one aspect, the opioid antagonist of the present invention administered is mu receptor-selective, delta receptor-selective or kappa receptor-selective. In a preferred embodiment, the opioid antagonist is mu receptor-selective e.g., naltrexone.

In another aspect, the opioid antagonist of the present invention is represented by the structure of the following formula:

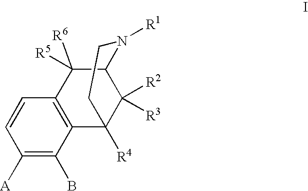

R¹ is selected from the group consisting of hydrogen, a substituted or unsubstituted, saturated or unsaturated aliphatic group, a substituted or unsubstituted, saturated or unsaturated alicyclic group, a substituted or unsubstituted aromatic group, a substituted or unsubstituted heteroaromatic group, or saturated or unsaturated heterocyclic group;

R² is selected from the group consisting of hydrogen, hydroxy, alkoxy, amino or substituted amino;

R³ and R⁴ are aliphatic;

R³ and R⁴ are taking together to form the following formula II:

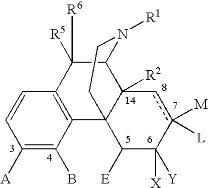

R⁵ and R⁶ are both hydrogen or taken together R⁵ and R⁶ are ═O;

A,B and E are independently selected from hydrogen, halogen, R¹, OR¹, SR¹, CONR³R⁴ and NR³R⁴; wherein R³ and R⁴ is independently selected from the group consisting of hydrogen, acyl, a substituted or unsubstituted, saturated or unsaturated aliphatic group, a substituted or unsubstituted, saturated or unsaturated alicyclic group, a substituted or unsubstituted aromatic group, a substituted or unsubstituted heteroaromatic group, saturated or unsaturated heterocyclic group; or can be taken together with the nitrogen atom to which they are attached to form a substituted or unsubstituted heterocyclic or heteroaromatic ring;

B and E are taken together to form the following formula III:

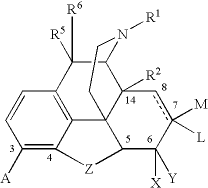

wherein Z is selected from O, S, or NR¹;
X and Y are independently selected from the group consisting of hydrogen, deuterium, halogen, nitrile, azide, R₁, OR₁, S(O)ₙR¹, —NR¹C(O)R¹, —NR¹C(O)NR³R⁴, —NR¹S(O)ₙR¹, —CONR³R⁴, and NR³R⁴;
or X and Y, taken together with the carbon atom to which they are attached, are selected from the group consisting of CO, C═CHR¹, C═NR¹, C═NOR¹, C═NO(CH₂)ₘR¹, C═NNHR¹, C═NNHCOR¹, C═NNHCONR¹R², C═NNHS(O)ₙR¹,or C═N—N═CHR¹;
R² and either X or Y taken together to form an additional sixth ring, which may be saturated or unsaturated;
L and M are independently selected from the group consisting of hydrogen, R₁, OR₁;
or L and M, taken together with the carbon atom to which they are attached, is selected from the group consisting of C═CHR¹, or a C₃-C₁₀ spiro-ftised carbocycle;
L and Y can be taken together to form a fused substituted or unsubstituted aryl or heteroaryl.

An “aliphatic group” is non-aromatic moiety that may contain any combination of carbon atoms, hydrogen atoms, halogen atoms, oxygen, nitrogen or other atoms, and optionally contain one or more units of unsaturation, e.g., double and/or triple bonds. An aliphatic group may be straight chained, branched or cyclic and preferably contains between about 1 and about 24 carbon atoms, more typically between about 1 and about 12 carbon atoms. In addition to aliphatic hydrocarbon groups, aliphatic groups include, for example, polyalkoxyalkyls, such as polyalkylene glycols, polyamines, and polyimines, for example. Such aliphatic groups may be further substituted.

The term “alkyl”, as used herein, refers to saturated, straight- or branched-chain hydrocarbon radicals containing between one or more carbon atoms. Examples of C₁-C₃ alkyl radicals include methyl, ethyl, propyl and isopropyl radicals; examples of C₁-C₆ alkyl radicals include, but are not limited to, methyl, ethyl, propyl, isopropyl, n-butyl, tert-butyl, sec-butyl, n-pentyl, neopentyl and n-hexyl radicals; and examples of C₁-C₁₂ alkyl radicals include, but are not limited to, ethyl, propyl, isopropyl, hexyl, heptyl, octyl, nonyl, decyl, undecyl, dodecyl radicals and the like.

The term “substituted alkyl,” as used herein, refers to an alkyl, such as a C₁-C₁₂ alkyl or C₁-C₆ alkyl group, substituted by one, two, three or more aliphatic substituents.

Suitable aliphatic substituents include, but are not limited to, —F, —Cl, —Br, —I, —OH, protected hydroxy, aliphatic ethers, aromatic ethers, oxo, —NO₂, —CN, —C₁-C₁₂-alkyl optionally substituted with halogen (such as perhaloalkyls), C₂-C₁₂-alkenyl optionally substituted with halogen, —C₂-C₁₂-alkynyl optionally substituted with halogen, —NH₂, protected amino, —NH—C₁-C₁₂-alkyl, —NH—C₂-C₁₂-alkenyl, —NH—C₂-C₁₂-alkenyl, —NH—C₃-C₁₂-cycloalkyl, —NH-aryl, —NH-heteroaryl, —NH-heterocycloalkyl, -dialkylamino, -diarylamino, -diheteroarylamino, —O—C₁-C₁₂-alkyl, —O—C₂-C₁₂-alkenyl, —O—C₂-C₁₂-alkynyl, —O—C₃-C₁₂-cycloalkyl, —O-aryl, —O-heteroaryl, —O-heterocycloalkyl, —C(O)—C₁-C₁₂-alkyl, —C(O)—C₂-C₁₂-alkenyl, —C(O)—C₂-C₁₂-alkynyl, —C(O)—C₃-C₁₂-cycloalkyl, —C(O)-aryl, —C(O)-heteroaryl, —C(O)-heterocycloalkyl, —CONH₂, —CONH—C₁-C₁₂-alkyl, —CONH—C₂-C₁₂-alkenyl, —CONH—C₂-C₁₂-alkynyl, —CONH—C₃-C₁₂-cycloalkyl, —CONH-aryl, —CONH-heteroaryl, —CONH-heterocycloalkyl, —CO₂-C₁—C₁₂-alkyl, —CO₂—C₂-C₁₂-alkenyl, —CO₂-C₂-C₁₂-alkynyl, —CO₂—C₃-C₁₂-cycloalkyl, —CO₂-aryl, —CO₂-heteroaryl, —CO₂-heterocycloalkyl, —OCO₂—C₁-C₁₂-alkyl, —OCO₂—C₂-C₁₂-alkenyl, —OCO₂—C₂-C₁₂-alkynyl, —OCO₂—C₃-C₁₂-cycloalkyl, —OCO₂-aryl, —OCO₂-heteroaryl, —OCO₂-heterocycloalkyl, —OCONH₂, —OCONH—C₁-C₁₂-alkyl, —OCONH—C₂-C₁₂-alkenyl, —OCONH—C₂-C₁₂-alkynyl, —OCONH—C₃-C₁₂-cycloalkyl, —OCONH-aryl, —OCONH-heteroaryl, —OCONH-heterocycloalkyl, —NHC(O)—C₁-C₁₂-alkyl, —NHC(O)—C₂-C₁₂-alkenyl, —NHC(O)—C₂-C₁₂-alkynyl, —NHC(O)—C₃-C₁₂-cycloalkyl, —NHC(O)-aryl, —NHC(O)-heteroaryl, —NHC(O)-heterocycloalkyl, —NHCO₂—C₁-C₁₂-alkyl, —NHCO₂—C₂-C₁₂-alkenyl, —NHCO₂—C₂-C₁₂-alkynyl, —NHCO₂—C₂-C₁₂-alkynyl, —NHCO₂—C₃-C₁₂-cycloalkyl, —NHCO₂-aryl, —NHCO₂-heteroaryl, —NHCO₂-heterocycloalkyl, —NHC(O)NH₂, NHC(O)NH—C₁-C₁₂-alkyl, —NHC(O)NH—C₂-C₁₂-alkenyl, —NHC(O)NH—C₂-C₁₂-alkynyl, —NHC(O)NH—C₃-C₁₂-cycloalkyl, —NHC(O)NH-aryl, —NHC(O)NH-heteroaryl, —NHC(O)NH-heterocycloalkyl, NHC(S)NH₂, NHC(S)NH—C₁-C₁₂-alkyl, —NHC(S)NH—C₂-C₁₂-alkenyl, —NHC(S)NH—C₂-C₁₂-alkynyl, —NHC(S)NH—C₃-C₁₂-cycloalkyl, —NHC(S)NH-aryl, —NHC(S)NH-heteroaryl, —NHC(S)NH-heterocycloalkyl, —NHC(NH)NH₂, NHC(NH)NH—C₁-C₁₂-alkyl, —NHC(NH)NH—C₂-C₁₂-alkenyl, —NHC(NH)NH—C₂-C₁₂-alkynyl, —NHC(NH)NH—C₃-C₁₂-cycloalkyl, —NHC(NH)NH-aryl, —NHC(NH)NH-heteroaryl, —NHC(NH)NH-heterocycloalkyl, NHC(NH)—C₁-C₁₂-alkyl, —NHC(NH)—C₂-C₁₂-alkenyl, —NHC(NH)—C₂-C₁₂-alkynyl, —NHC(NH)—C₃-C₁₂-cycloalkyl, —NHC(NH)-aryl, —NHC(NH)-heteroaryl, —NHC(NH)-heterocycloalkyl, —C(NH)NH—C₁-C₁₂-alkyl, —C(NH)NH—C₂-C₁₂-alkenyl, —C(NH)NH—C₂-C₁₂-alkynyl, —C(NH)NH—C₃-C₁₂-cycloalkyl, —C(NH)NH-aryl, —C(NH)NH-heteroaryl, —C(NH)NH-heterocycloalkyl, —S(O)—C₁-C₁₂-alkyl, —S(O)—C₂-C₁₂-alkenyl, —S(O)—C₂-C₁₂-alkynyl, —S(O)—C₃-C₁₂-cycloalkyl, —S(O)-aryl, —S(O)-heteroaryl, —S(O)-heterocycloalkyl —SO₂NH₂, —SO₂NH—C₁-C₁₂-alkyl, —SO₂NH—C₂-C₁₂-alkenyl —SO₂NH—C₂-C₁₂-alkynyl, —SO₂NH—C₃-C₁₂-cycloalkyl, —SO₂NH-aryl, —SO₂NH-heteroaryl, —SO₂NH-heterocycloalkyl, —NHSO₂—C₁-C₁₂-alkyl, —NHSO₂—C₂-C₁₂-alkenyl, —NHSO₂—C₂-C₁₂-alkynyl, —NHSO₂—C₃-C₁₂-cycloalkyl, —NHSO₂-aryl, —NHSO₂-heteroaryl, —NHSO₂-heterocycloalkyl, —CH₂NH₂, —CH₂SO₂CH₃, -aryl, -arylalkyl, -heteroaryl, -heteroarylalkyl, -heterocycloalkyl, —C₃-C₁₂-cycloalkyl, polyalkoxyalkyl, polyalkoxy, -methoxymethoxy, -methoxyethoxy, —SH, —S—C₁-C₁₂-alkyl, —S—C₂-C₁₂-alkenyl, —S—C₂-C₁₂-alkynyl, —S—C₃-C₁₂-cycloalkyl, —S-aryl, —S-heteroaryl, —S-heterocycloalkyl, or methylthiomethyl. It is understood that the aryls, heteroaryls, alkyls and the like can be further substituted.

The term “alkenyl”, as used herein, denotes a monovalent group derived from a hydrocarbon moiety containing from two to twelve or two to six carbon atoms having at least one carbon-carbon double bond by the removal of a single hydrogen atom. Alkenyl groups include, but are not limited to, for example, ethenyl, propenyl, butenyl, 1-methyl-2-buten-1-yl, alkadienes and the like.

The term “alkynyl”, as used herein, denotes a monovalent group derived from a hydrocarbon moiety containing from two to twelve or two to six carbon atoms having at least one carbon-carbon triple bond by the removal of a single hydrogen atom. Representative alkynyl groups include, but are not limited to, for example, ethynyl, 1-propynyl, 1-butynyl, and the like.

The term “aryl” or “aromatic,” as used herein, refers to a mono- or bicyclic carbocyclic ring system having one or two aromatic rings including, but not limited to, phenyl, naphthyl, tetrahydronaphthyl, indanyl, idenyl and the like.

Aromatic substituents include, but are not limited to, —F, —Cl, —Br, —I, —OH, protected hydroxy, aliphatic ethers, aromatic ethers, oxo, —NO₂, —CN, —C₁-C₁₂-alkyl optionally substituted with halogen (such as perhaloalkyls), C₂-C₁₂-alkenyl optionally substituted with halogen, —C₂-C₁₂-alkynyl optionally substituted with halogen, —NH₂, protected amino, —NH—C₁-C₁₂-alkyl, —NH—C₂-C₁₂-alkenyl, —NH—C₂-C₁₂-alkenyl, —NH—C₃-C₁₂-cycloalkyl, —NH-aryl, —NH-heteroaryl, —NH-heterocycloalkyl, -dialkylamino, -diarylamino, -diheteroarylamino, —O—C₁-C₁₂-alkyl, —O—C₂-C₁₂-alkenyl, —O—C₂-C₁₂-alkynyl, —O—C₃-C₁₂-cycloalkyl, —O-aryl, —O-heteroaryl, —O-heterocycloalkyl, —C(O)—C₁-C₁₂-alkyl, —C(O)—C₂-C₁₂-alkenyl, —C(O)—C₂-C₁₂-alkynyl, —C(O)—C₃-C₁₂-cycloalkyl, —C(O)-aryl, —C(O)-heteroaryl, —C(O)-heterocycloalkyl, —CONH₂, —CONH—C₁-C₁₂-alkyl, —CONH—C₂-C₁₂-alkenyl, —CONH—C₂-C₁₂-alkynyl, —CONH—C₃-C₁₂-cycloalkyl, —CONH-aryl, —CONH-heteroaryl, —CONH-heterocycloalkyl, —CO₂—C₁-C₁₂-alkyl, —CO₂—C₂-C₁₂-alkenyl, —CO₂—C₂-C₁₂-alkynyl, —CO₂—C₃-C₁₂-cycloalkyl, —CO₂-aryl, —CO₂-heteroaryl, —CO₂-heterocycloalkyl, —OCO₂—C₁-C₁₂-alkyl, —OCO₂—C₂-C₁₂-alkenyl, —OCO₂—C₂-C₁₂-alkynyl, —OCO₂—C₃-C₁₂-cycloalkyl, —OCO₂-aryl, —OCO₂-heteroaryl, —OCO₂-heterocycloalkyl, —OCONH₂, —OCONH—C₁-C₁₂-alkyl, —OCONH—C₂-C₁₂-alkenyl, —OCONH—C₂-C₁₂-alkynyl, —OCONH—C₃-C₁₂-cycloalkyl, —OCONH-aryl, —OCONH-heteroaryl, —OCONH-heterocycloalkyl, —NHC(O)—C₁-C₁₂-alkyl, —NHC(O)—C₂-C₁₂-alkenyl, —NHC(O)—C₂-C₁₂-alkynyl, —NHC(O)—C₃-C₁₂-cycloalkyl, —NHC(O)-aryl, —NHC(O)-heteroaryl, —NHC(O)-heterocycloalkyl, —NHCO₂—C₁-C₁₂-alkyl, —NHCO₂—C₂-C₁₂-alkenyl, —NHCO₂—C₂-C₁₂-alkynyl, —NHCO₂—C₃-C₁₂-cycloalkyl, —NHCO₂-aryl, —NHCO₂-heteroaryl, —NHCO₂-heterocycloalkyl, —NHC(O)NH₂, NHC(O)NH—C₁-C₁₂-alkyl, —NHC(O)NH—C₂-C₁₂-alkenyl, —NHC(O)NH—C₂-C₁₂-alkynyl, —NHC(O)NH—C₃-C₁₂-cycloalkyl, —NHC(O)NH-aryl, —NHC(O)NH-heteroaryl, —NHC(O)NH-heterocycloalkyl, NHC(S)NH₂, NHC(S)NH—C₁-C₁₂-alkyl, —NHC(S)NH—C₂-C₁₂-alkenyl, —NHC(S)NH—C₂-C₁₂-alkynyl, —NHC(S)NH—C₃-C₁₂-cycloalkyl, —NHC(S)NH-aryl, —NHC(S)NH-heteroaryl, —NHC(S)NH-heterocycloalkyl, —NHC(NH)NH₂, NHC(NH)NH—C₁-C₁₂-alkyl, —NHC(NH)NH—C₂-C₁₂-alkenyl, —NHC(NH)NH—C₂-C₁₂-alkynyl, —NHC(NH)NH—C₃-C₁₂-cycloalkyl, —NHC(NH)NH-aryl, —NHC(NH)NH-heteroaryl, —NHC(NH)NH-heterocycloalkyl, NHC(NH)—C₁-C₁₂-alkyl, —NHC(NH)—C₂-C₁₂-alkenyl, —NHC(NH)—C₂-C₁₂-alkynyl, —NHC(NH)—C₃-C₁₂-cycloalkyl, —NHC(NH)-aryl, —NHC(NH)-heteroaryl, —NHC(NH)-heterocycloalkyl, —C(NH)NH—C₁-C₁₂-alkyl, —C(NH)NH—C₂-C₁₂-alkenyl, —C(NH)NH—C₂-C₁₂-alkynyl, —C(NH)NH—C₃-C₁₂-cycloalkyl, —C(NH)NH-aryl, —C(NH)NH-heteroaryl, —C(NH)NH-heterocycloalkyl, —S(O)—C₁-C₁₂-alkyl, —S(O)—C₂-C₁₂-alkenyl, —S(O)—C₂-C₁₂-alkynyl, —S(O)—C₃-C₁₂-cycloalkyl, —S(O)-aryl, —S(O)-heteroaryl, —S(O)-heterocycloalkyl —SO₂NH₂, —SO₂NH—C₁-C₁₂-alkyl, —SO₂NH—C₂-C₁₂-alkenyl, —SO₂NH—C₂-C₁₂-alkynyl, —SO₂NH—C₃-C₁₂-cycloalkyl, —SO₂NH-aryl, —SO₂NH-heteroaryl, —SO₂NH-heterocycloalkyl, —NHSO₂—C₁-C₁₂-alkyl, —NHSO₂—C₂-C₁₂-alkenyl, —NHSO₂—C₂-C₁₂-alkynyl, —NHSO₂—C₃-C₁₂-cycloalkyl, —NHSO₂-aryl, —NHSO₂-heteroaryl, —NHSO₂-heterocycloalkyl, —CH₂NH₂, —CH₂SO₂CH₃, -aryl, -arylalkyl, -heteroaryl, -heteroarylalkyl, -heterocycloalkyl, —C₃-C₁₂-cycloalkyl, polyalkoxyalkyl, polyalkoxy, -methoxymethoxy, -methoxyethoxy, —SH, —S—C₁-C₁₂-alkyl, —S—C₂-C₁₂-alkenyl, —S—C₂-C₁₂-alkynyl, —S—C₃-C₁₂-cycloalkyl, —S-aryl, —S-heteroaryl, —S-heterocycloalkyl, or methylthiomethyl. It is understood that the aryls, heteroaryls, alkyls and the like can be further substituted.

The term “arylalkyl,” as used herein, refers to an aryl group attached to the parent compound via an alkyl residue. Examples include, but are not limited to, benzyl, phenethyl and the like.

The term “heteroaryl” or “heteroaromatic,” as used herein, refers to a mono-, bi-, or tri-cyclic aromatic radical or ring having from five to ten ring atoms of which at least one ring atom is selected from S, O and N; zero, one or two ring atoms are additional heteroatoms independently selected from S, O and N; and the remaining ring atoms are carbon, wherein any N or S contained within the ring may be optionally oxidized. Heteroaryl includes, but is not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl, and the like. The heteroaromatic ring may be bonded to the chemical structure through a carbon or hetero atom.

The term “cycloalkyl,” as used herein, denotes a monovalent group derived from a monocyclic or bicyclic saturated carbocyclic ring compound by the removal of a single hydrogen atom. Examples include, but not limited to, cyclopropyl, cyclobutyl, cyclopentyl, cyclohexyl, bicyclo[2.2.1]heptyl, and bicyclo[2.2.2]octyl.

The term “heterocycloalkyl,” as used herein, refers to a non-aromatic 5-, 6- or 7-membered ring or a bi- or tri-cyclic group fused system, where (i) each ring contains between one and three heteroatoms independently selected from oxygen, sulfur and nitrogen, (ii) each 5-membered ring has 0 to 1 double bonds and each 6-membered ring has 0 to 2 double bonds, (iii) the nitrogen and sulfur heteroatoms may optionally be oxidized, (iv) the nitrogen heteroatom may optionally be quatemized, (iv) any of the above rings may be fused to a benzene ring, and (v) the remaining ring atoms are carbon atoms which may be optionally oxo-substituted. Representative heterocycloalkyl groups include, but are not limited to, [1,3]dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, quinoxalinyl, pyridazinonyl, and tetrahydrofuryl.

The term “hydroxy protecting group,” as used herein, refers to a labile chemical moiety which is known in the art to protect a hydroxyl group against undesired reactions during synthetic procedures. After said synthetic procedure(s) the hydroxy protecting group as described herein may be selectively removed. Hydroxy protecting groups as known in the are described generally In T. H. Greene and P. G. M. Wuts, Protective Groups in Organic Synthesis, 3rd edition, John Wiley & Sons, New York (1999). Examples of hydroxyl protecting groups include benzyloxycarbonyl, 4-nitrobenzyloxycarbonyl, 4-bromobenzyloxycarbonyl, 4-methoxybenzyloxycarbonyl, methoxycarbonyl, tert-butoxycarbonyl, isopropoxycarbonyl, diphenylmethoxycarbonyl, 2,2,2-trichloroethoxycarbonyl, 2-(trimethylsilyl)ethoxycarbonyl, 2-furfuryloxycarbonyl, allyloxycarbonyl, acetyl, formyl, chloroacetyl, trifluoroacetyl, methoxyacetyl, phenoxyacetyl, benzoyl, methyl, t-butyl, 2,2,2-trichloroethyl, 2-trimethylsilyl ethyl, 1,1-dimethyl-2-propenyl, 3-methyl-3-butenyl, allyl, benzyl, para-methoxybenzyldiphenylmethyl, triphenylmethyl (trityl), tetrahydrofuryl, methoxymethyl, methylthiomethyl, benzyloxymethyl, 2,2,2-triehloroethoxymethyl, 2-(trimethylsilyl)ethoxymethyl, methanesulfonyl, para-toluenesulfonyl, trimethylsilyl, triethylsilyl, triisopropylsilyl, and the like. Preferred hydroxyl protecting groups for the present invention are acetyl (Ac or —C(O)CH₃), benzoyl (Bn or —C(O)C₆H₅), and trimethylsilyl (TMS or —Si(CH₃)₃).

The term “protected hydroxy,” as used herein, refers to a hydroxy group protected with a hydroxy protecting group, as defined above, including benzoyl, acetyl, trimethylsilyl, triethylsilyl, methoxymethyl groups, for example.

The term “amino protecting group,” as used herein, refers to a labile chemical moiety which is known in the art to protect an amino group against undesired reactions during synthetic procedures. After said synthetic procedure(s) the amino protecting group as described herein may be selectively removed. Amino protecting groups as known in the are described generally In T. H. Greene and P. G. M. Wuts, Protective Groups in Organic Synthesis, 3rd edition, John Wiley & Sons, New York (1999). Examples of amino protecting groups include, but are not limited to, t-butoxycarbonyl, 9-fluorenylmethoxycarbonyl, benzyloxycarbonyl, and the like.

The term “protected amino,” as used herein, refers to an amino group protected with an amino protecting group as defined above.

The term “acyl” includes residues derived from acids, including but not limited to carboxylic acids, carbamic acids, carbonic acids, sulfonic acids, and phosphorous acids. Examples include aliphatic carbonyls, aromatic carbonyls, aliphatic sulfonyls, aromatic sulfinyls, aliphatic sulfinyls, aromatic phosphates and aliphatic phosphates.

In a preferred embodiment, the compound can be naltrexone or its derivatives: 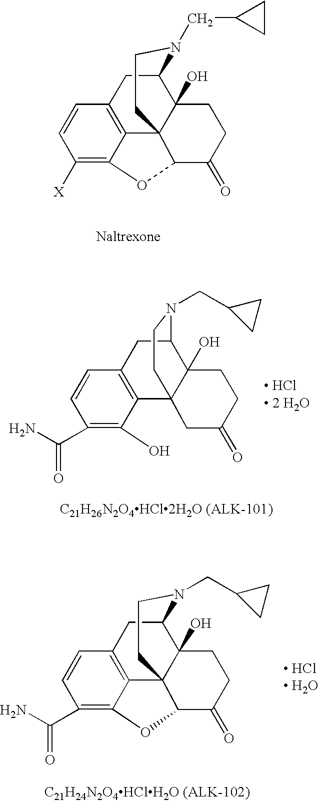
The compounds (including other salts, solvates, hydrates or free bases thereof) can be prepared using the procedures described in PCT WO02/36573 which is incorporated herein by reference.

In another aspect, the second compound of the present invention is selected from the group consisting of a GABA B agonist e.g., baclofen; an NMDA antagonist e.g., memantine; a serotonin antagonist e.g., buspirone, ondansetron or granisetron; and a cannabinoid antagonist e.g., SR-141716A or AM-251.

In yet another aspect, the opioid antagonist of the present invention is administered in a daily dose ranging from about from about 1 mg to about 500 mg and the second compound in said composition is administered in a daily dose ranging from about from about 1 mg to about 500 mg.

In still another aspect, the composition of the present invention further comprises a sustained release carrier such that the dosage form is administrable on a twice-a-day or on a once-a-day basis. In another aspect, the sustained release carrier causes said composition to be released over a time period of about 8 to about 24 hours when orally administered to a human patient. In still another aspect, the sustained release carrier is formulated as a tablet, capsule, pill, lozenge or potion.

In a further aspect, the symptoms ameliorated or eliminated by the composition of the present invention include anxiety, nausea, excitability, insomnia, craving, irritability, impulsivity, anger or rage.

In yet a further aspect, the therapeutic response being achieved by the composition of the present invention is a synergistic or additive effect.

In still a further aspect, the brain reward system disorder being treated by the compositions of the present invention is selected from the group comprising pathological gambling, compulsive alcohol consumption, compulsive over-eating and obesity, compulsive smoking, and drug addiction.

In yet another aspect, the present invention relates to an orally administrable dosage form containing the pharmaceutical composition, wherein said dosage form provides a once daily dosing for therapeutic relief of at least one symptom of a brain reward system disorder.

The invention also relates to a method for the treatment of brain reward system disorders comprising concurrently administering to a subject in need of treatment a therapeutically effective amount of: (i) a first compound comprising an opioid antagonist or a pharmaceutically acceptable salt, isomer, prodrug, analog, metabolite or derivative thereof; and (ii) and a second compound effective to ameliorate or eliminate at least one symptom of an brain reward system disorder; wherein the combined therapy potentiates the therapeutic response compared to treatment of either compound as monotherapy.

The invention still further relates to a method for changing brain reward system disorders behavior of a subject suffering from withdrawal symptoms associated with alcohol abuse comprising administering a therapeutically effective amount of: (i) a first compound comprising an opioid antagonist or a pharmaceutically acceptable salt, isomer, prodrug, analog, metabolite or derivative thereof; and (ii) and a second compound effective to ameliorate or eliminate at least one symptom of a brain reward system disorder; wherein the combined therapy potentiates the therapeutic response compared to treatment of either compound as monotherapy.

The invention also relates to a sustained-release formulation for the treatment of brain reward system disorders comprising concurrently administering to a subject in need of treatment a therapeutically effective amount of: (i) a first compound comprising an opioid antagonist or a pharmaceutically acceptable salt, isomer, prodrug, analog, metabolite or derivative thereof; and (ii) and a second compound effective to ameliorate or eliminate at least one symptom of brain reward system disorders; wherein the combined therapy potentiates the therapeutic response compared to treatment of either compound as monotherapy.

The invention further relates pharmaceutical kit comprising an oral dosage form of a first compound comprising an opioid antagonist and a second compound that effectively ameliorates or eliminates at least one symptom of a brain reward system disorder.

### BRIEF DESCRIPTION OF THE DRAWINGS

FIG. 1: Mean Plasma Concentration-Time Profile of RDC-0313-01 and Naltrexone in Rats Following SC Administration (0.1 mg/kg).

FIG. 2: Mean Plasma Concentration-Time Profile of RDC-0313-01, RDC-5818-01 and Naltrexone in Rats Following PO Administration (10 mg/kg).

FIG. 3: Efficacy and Potency of Opioid Antagonists on Blockade of Morphine-Induced Analgesia (15 mg/kg, IP, 30 Minutes Following Opioid Antagonist Administration, SC).

FIG. 4: Duration of Action of Opioid Antagonists on Blockade of Morphine-Induced Analgesia (15 mg/kg, IP, 30 Minutes Prior to Hot Plate Test).

FIG. 5: Lack of Tolerance of Opioid Antagonists Following Five days of Repeated Dosing (SC).

FIG. 6: Naltrexone Suppresses the Self-Administration of Ethanol in a Dose-Dependent Manner.

FIG. 7: Naltrexone's Effects on Drinking is Specific for Ethanol.

FIG. 8: Effect of Route of Administration on the Self-Administration of Ethanol

FIG. 9: Synergistic Effect of Coadministration of AM-251 with Naltrexone on the Self-Administration of Ethanol.

FIG. 10: Lack of Tolerance Following Repeated Dosing (5 Days) of AM-251 with Naltrexone on the Self-Administration of Ethanol.

FIG. 11: Additive Effects of the Coadministration of Baclofen with Naltrexone on the Self-Administration of Ethanol.

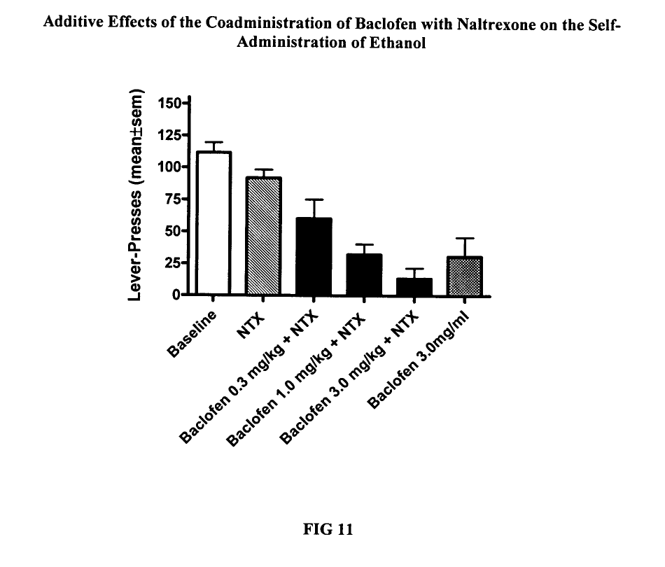

FIG. 12: Effect of Baclofen Alone on the Self-Administration of Ethanol.

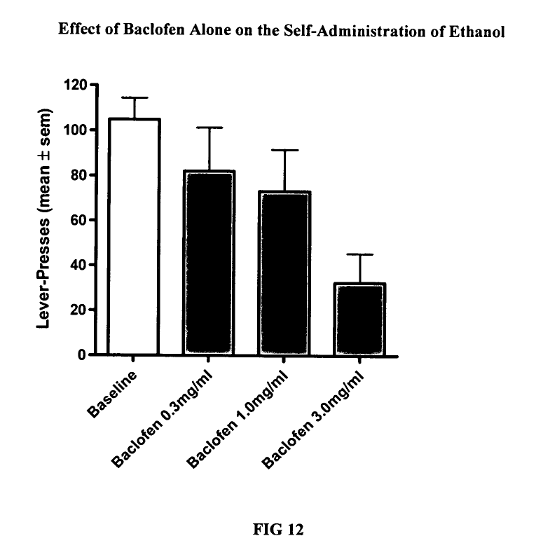

FIG. 13: Lack of Tolerance Following Repeated Dosing (5 Days) of the Coadministration of Baclofen with Naltrexone on the Self-Administration of Ethanol.

### DETAILED DESCRIPTION OF THE INVENTION

The present invention is directed to a combination treatment of an opioid antagonist e.g., naltrexone and a second compound selected from the group consisting of a GABA B agonist, an NMDA antagonist, a serotonin antagonist, and a cannabinoid antagonist for the successful treatment of a brain reward system disorder. The brain reward system is a neural network in the middle of the brain that prompts good feelings in response to certain behaviors. Dopamine is commonly associated with the ‘pleasure system’ of the brain, providing feelings of enjoyment and reinforcement to motivate us to do, or continue doing, certain activities. Dopamine is released (particularly in areas such as the nucleus accumbens and striatum) by naturally rewarding experiences such as food, sex, use of certain drugs and neutral stimuli that become associated with them. Brain reward system disorders are characterized by an inability to refrain from repeatedly engaging in an addictive behavior e.g., nicotine/tobacco, alcohol and/or drug abuse, or compulsive behaviors e.g., pathological gambling, and/or compulsive over-eating and obesity. Individuals who abstain from an addictive and compulsive or excessive behavior often experience cravings, withdrawal symptoms and negative drug side effects. The present invention is also based a combination treatment produces a synergistic or additive effect on a disorder associated with the brain reward system. For example, the combined effect of administering two therapeutic compounds e.g., naltrexone plus a second compound described herein, produces an overall response that is greater than the sum of the two individual effects. Furthermore, the synergistic or additive effect of the combined therapy allows for a lower dosing regime than that currently available in the market place for a monotherapy. In turn, the compounds and methods of the present invention effectively reduce the cravings, withdrawal symptoms and negative drug side effects associated with a monotherapy. As such, patient compliance is greatly increased, thereby decreasing relapse of a brain reward system disorder. The present invention provides compositions and methods for treating a subject associated with a brain reward system disorder, in particular effective treatments against the cravings, withdrawal symptoms and negative drug side effects.

In order that the present invention may be more readily understood, certain terms are first defined. Additional definitions are set forth throughout the detailed description.

As used herein, the term “brain reward system disorders” refer to diseases or disorders associated with a subject's inability to refrain participating in compulsive, excessive or addictive behavior associated with dopaminergic activation. Subjects afflicted with a brain reward system disorder receive a pleasurable “high” which reinforces or motivates a subject to continue engaging in the compulsive, excessive or addictive behavior. As dopamine levels are increased a subject engages in the activity even more vigorously, taking greater risks to achieve the same pleasurable “high”. Non-limiting examples of brain reward system disorders which can be treated by the present invention include the following: pathological gambling, compulsive alcohol consumption, compulsive over-eating and obesity, compulsive smoking, and drug addiction.

As used herein, “drug addiction” refers to a physical and/or psychological tolerance to a drug, e.g., nicotine, alcohol, heroine, cocaine, opium, codeine, LSD, methamphetamine, and crack. Tolerance means a need to increase the dose progressively in order to produce the effect originally achieved by smaller amounts.

As used herein, “an excessive or compulsive behavior” refers to a psychological tolerance to an unhealthy stimulus e.g., pathological gambling, “checking” behaviors, compulsive shopping, compulsive working, compulsive exercising, compulsive lying, sexual compulsion, self-abuse/cutting, kleptomania. Tolerance means a need to increase the activity progressively e.g., take greater risks, in order to achieve the same level of excitement.

As used herein, an “eating disorder” refers to compulsive overeating, obesity or severe obesity. Obesity means body weight of 20% over standard height-weight tables. Severe obesity means over 100% overweight.

As used herein “pathological gambling” refers to a condition characterized by a preoccupation with gambling. Similar to psychoactive substance abuse, its effects include development of tolerance with a need to gamble progressively larger amounts of money, withdrawal symptoms, and continued gambling despite severe negative effects on family and occupation.

As used herein, the term “compulsive smoking” refers to a condition characterized by an addiction to nicotine in tobacco products e.g., cigarettes and cigars. Addiction to nicotine is often accompanied by an oral fixation, wherein the smoker enjoys holding and sucking on cigarettes. This oral fixation allows smokers something to do with their hands thereby making quitting difficult.

As used herein the term “compulsive alcohol consumption” refers to a condition wherein the subject's continued excessive use of alcoholic drinks results in a loss of control over the subject's drinking. A subject will continue drinking despite its interference with some vital area of her or his life such as family, friends, job, school or health.

Individuals suffering from a brain reward system disorder are identified by the presence of any one or more of a number of undesired symptoms upon abstinence of the unhealthy stimulus e.g., cravings and withdrawal or withdrawal-like symptoms. Subjects suffering from a brain reward system disorder often experience a physical dependence and/or psychological dependence to the addictive or excessive or compulsive behavior. Physical dependency occurs when a drug e.g, tobacco, nicotine, heroine, etc., has been used habitually and the body has become accustomed to its effects. The person must then continue to use the drug in order to feel normal, or its absence will trigger the symptoms of withdrawal. Psychological dependency occurs when an addict and/or a subject afflicted with an excessive or compulsive behavior has used or engaged in the behavior habitually and the mind has become emotionally reliant of its harmful effects, either to elicit pleasure or relieve pain, and does not feel capable of functioning without it. Its absence produces intense cravings, which are often brought on or magnified by stress, followed by withdrawal or withdrawal like symptoms. In addition, treatment regimes often produce negative drug side effects e.g., nausea which makes compliance challenging.

The term “cravings” as described herein, refers to an uncontrollable desire or urge whether conscious or subconscious to engage in an addictive or compulsive or excessive behavior.

The term “withdrawal” as described herein, refers to the physical or psychological state experienced when certain harmful stimulus e.g., brain reward system disorder are discontinued.

The term “ameliorating or eliminating at least one symptom of a brain reward symptom disorder” refers to preventing, partially or totally, symptoms often associated with treatment of a brain reward system disorder (e.g., cravings, withdrawal and/or drug side effects) including but not limited to feelings of jumpiness or nervousness; feeling of shakiness; anxiety; irritability; or being excited; difficulty in thinking clearly; bad dreams; emotional volatility; rapid emotional changes; depression; fatigue; headache (generally pulsating); sweating (especially palms of the hands and face); nausea; vomiting; loss of appetite; insomnia or sleep difficulty; paleness; rapid heart rate (palpitations); eyes, especially pupils, different size (enlarged, dilated pupils); clammy skin; abnormal movement of the eyelids; state of confusion and hallucinations (also called delirium tremens); agitation; fever; convulsions; “black outs.” (Source: National Institute of Health).

In one embodiment, the current invention relates to a combined use of an opioid antagonist e.g., naltrexone with a second compound consisting of a GABA B agonist, an NMDA antagonist, a serotonin antagonist, and a cannabinoid antagonist to treat a brain reward system disorder. The pharmaceutical composition as described herein relates to a combination of an effective amount of the opioid antagonist, preferably naltrexone or mixtures thereof, and at least one second compound, preferably baclofen, memantine, buspirone, ondansetron, gabapentin, SR-141716A and AM-251 or mixtures thereof.

The term “combination” as in the phrase “a first compound in combination with a second compound” includes co-administration of a first agent and a second agent, which for example may be dissolved or intermixed in the same pharmaceutically acceptable carrier. The term concurrently administered when referring to compound (i) and compound (ii) of the present invention, is meant that each compound may be administered at the same time or sequentially in any order at different points in time, however if not administered at the same time, they should be administered sufficiently closely in time so as to provide the desired treatment effect. Preferably, all components are administered at the same time, and if not administered at the same time, preferably they are all administered less than one hour apart from one another.

The term “synergistic” and/or “additive” effect as used herein refers to the combined effect of administering two therapeutic compounds where the overall response is greater than the sum of the two individual effects. The term synergy or additive also refers to the combined effect of administering an amount of one compound that, when administered as monotherapy, produces no measurable response but, when administered in combination with another therapeutic compound, produces an overall response that is greater than that produced by the second compound alone.

The term “treating of a brain reward system disorder” refers to reversing, alleviating, inhibiting the progress of, or preventing a brain reward system disease or disorder, or preventing one or more symptoms (e.g., craving, withdrawal and/or drug side effects) of a brain reward system disease or disorder. The term “treatment”, as used herein, refers to the act of treating, as defined immediately above.

Opioid antagonist as referred to herein are compounds or compositions which serve to block the action of endogenous or exogenous opioid compounds on narcotic receptors or narcotic receptor subtypes in the brain or periphery. Opioid antagonists of the present invention are those that bind with high specificity to mu, delta or kappa receptors. Representative opioid antagonists and inverse agonists include at least one of the following: naltrexone (marketed in 50 mg dosage forms from Du Pont Pharrna as ReVia™ or Trexan™), naloxone (marketed as Narcane™, NALOXONE/PENTAZOCINE™ from Pharma Pac), nalmefene, methylnaltrexone, naloxone methiodide, nalorphine, naloxonazine, nalide, nalmexone, nalbuphine, nalorphine dinicotinate, naltrindole (NTI), naltrindole isothiocyanate, (NTII), naltriben (NTB), nor-binaltorphimine (nor-BNI), b-funaltrexamine (b-FNA), BNTX, cyprodime, ICI-174,864, LY117413, MR2266, NE-100, SSR 125329, MS 377, J113397, E6276, CJ15208, LY255582 or an opioid antagonist having the same pentacyclic nucleus as nalmefene, naltrexone, buprenorphine, levorphanol, meptazinol, pentazocine, dezocine, or their pharmacologically effective esters or salts. In preferred embodiments, the opioid antagonist of the present invention is naltrexone.

In one embodiment the naltrexone is naltrexone hydrochloride (HCL) which is available generically and under the trade name ReVia™ or Trexan™. Naltrexone is currently available in oral tablet form and is approved by the U.S. Food and Drug Administration (FDA) for the treatment of alcoholism as well as heroin and opium addiction. While not being held to one particular theory, it is believed that opioid antagonist act by blocking the positive reinforcing effects associated with the release of dopamine which results from the release of endogenous opioids.

In general, naltrexone is used in the treatment alcoholism. Most patients take naltrexone for 12 weeks or more. In general, the treatment involves taking a prescribed course of naltrexone tablets for up to one year. These tablets are taken by mouth, one a day, every couple of days at higher does. Generally, the doctor may initially monitor the patient's progress quite closely. Naltrexone's effects on blocking opioids occur shortly after taking the first dose. Findings to date suggest that the effects of naltrexone in helping patients remain abstinent and avoid relapse of alcoholism.

It is known that some patients have adverse clinical manifestations like nausea, headache, constipation, dizziness, nervousness, insomnia, drowsiness, anxiety and the like. Naltrexone adverse clinical manifestations, predominately nausea, have been severe enough to discontinue medication in 5-10% of the patients prescribed it as a treatment for alcoholism. If a patient gets any of these adverse clinical manifestations and consults the doctor, the doctor may be forced to change the treatment or suggest other ways to deal with the adverse clinical manifestations. Often instead of seeing a doctor, the patient will “self-treat” by skipping doses or stopping doses altogether.

Combined treatment of an opioid antagonist and a second compound consisting of a GABA B agonist, an NMDA antagonist, a serotonin antagonist, and a cannabinoid antagonist can result in the successful treatment of a brain reward system disorder, in particular the treating the cravings, withdrawal symptoms associated with abstention and the negative drug side effects associated with a monotherapy.

A “GABA-ergic” agent is an agent that exerts a GABA-like effect, and include GABA-agonists and agents that have effects like GABA-agonists Representative GABA agonists, antagonists and modulators include at least one of the following: muscimol, baclofen, APPA, APMPA, CaCa, valproic acid, indiplon, ocinaplon, zalepon, CGP44532, RO15-4513, RO19-4603, pregabaline, L-655,708, RY-23, AVE-1876, RU 34000, flumazenil, NGD96-3, NG2-73, CGP7930, CGP13501, GS39783, a neuroactive steroid, a barbiturate, a benzodiazepine, gabapentin, tigabine, or vigabatrin. In preferred embodiments, the GABA-ergic agonist of the present invention is baclofen.

An N-methyl-D-aspartate (NMDA) antagonist is an agent which binds to NMDA receptors and/or block any of the sites that bind glycine, glutamate, NMDA or phencyclidine (PCP). Blocking the NMDA receptor sites has the effect of preventing the creation of an action potential in the cell. NMDA receptor antagonists include those compounds that preferentially bind to NMDA receptors, but may also have other activities. Representative modulators of glutamate receptors and NMDA antagonists include at least one of the following: dextromethorphan, dextrophan, dextropropoxyphene, dizocilpine, Cerestat™ (CNS-1102), ketamine, ketobemidone, MPEP, MTEP, YM-298198, LY354,740, CGP 37849, L-701-324, ifenprodil, perzinfotel, CGX-1007, UK-240455, besonprodil, AZ D 4282, SIB 1893, RO-0256981, PRE703, Licostinel™ (ACEA 1021), Selfotel™ (CGS-19755), D-CPP-ene (SDZ EAA 494; EAA-494-Leppik), memantine ((1-amino-3,5-dimethyl adamantane) is an analog of 1-aminocyclohexane (amantadine) and is disclosed in U.S. Pat. Nos. 4,122,193; 4,273,774; 5,061,703; and 5,614,560), methadone, ibogaine, LY235,959, naphthalenesulfonamide, neramexane ((1-amino-1,3,3,5,5-pentamethylcyclohexane) is also a derivative of 1-aminocyclohexane, and is disclosed in U.S. Pat. No. 6,034,134), phencyclidine and trifluoperazine. In preferred embodiments, the NMDA antagonist of the present invention is memantine.

As used herein the term, “serotonin antagonist” refers to drugs that bind to but do not activate serotonin receptors, thereby blocking the actions of serotonin or serotonin agonists. Representative serotonin agonists, antagonists, reuptake inhibitors and modulators include at least one of the following: alosetron, ondansetron, granisetron, bemesetron, eplivanserine (SR-46349B), M-100907, deramciclane, agomelatine (S-20098), elazasonan (CP-448,187), pruvanserin (EMD-281014), AVE 8488, asenapine (ORG 5222), zomaril (iloperidone), MN-305, valazodone, bifeprunox (DU-127090), buspirone, ritanseron, PRX-00023, APD125, geperone ER, paliperidone, ACP-103, OPC-14523 (VPI-013), clomipram, SEP-225289, DOV102,677, DOV216,303, DOV21,947, doxepin, GW-372475 (NS2359), ICS205-930, an SSRI (fluoxetine, citalopram, sertaline). In preferred embodiments the serotonin antagonist is ondansetron and granisetron.

As used herein the term, “cannabinoid antagonist” refers to drugs that bind to and block cannabinoid receptors. Representative cannabinoid antagonist and inverse agonists include at least one of the following: rimonabant (SR141716A Sanofi Synthelabo) SR-147778 (Rinaldi-Carmona, et al., Life Sci., 56:1941-1947 (1995)), AM-251, AM-281, CP-272,871, NIDA-41020, NESS 0327, O-1248, O-1803, SLV-326, SLV-319, AVE-1625 and CP-945598. In preferred embodiments the cannabinoid antagonist is SR-141716A and AM-251.

Dosage and Route of Administration

Suitable daily oral dosages for the active agents described herein are on the order of about 0.01 mg to about 1,000 mg of each active agent described herein. Desirably, each oral dosage contains from 0.01 to 1,000 mg, particularly 0.01, 0.05, 0.1, 0.2, 0.5, 1.0, 2.5, 5, 10, 15, 20, 25, 30, 40, 50, 75, 100, 125, 150, 175, 200, 225, 250, 500, 750, 850 and 1,000 milligrams of each active ingredient in the composition of the present invention (e.g. each opioid antagonist, each GABA B agonist, each NMDA antagonist, each serotonin antagonist, and each cannabinoid antagonist) administered for the treatment of a brain reward disorder. Dosage regimen may be adjusted to provide the optimal therapeutic response. The specific dose level for any particular patient will vary depending upon a variety of factors, including but not limited to, the activity of the specific compound employed; the age, body weight, general health, sex and diet of the patient; the time of administration; the rate of excretion; drug combination; the severity of the particular disease being treated; and the form of administration. Typically, in vitro dosage-effect results provide useful guidance on the proper doses for patient administration. Studies in animal models are also helpful. The considerations for determining the proper dose levels are well known in the art.

The weight ratio of the active agents in the in the instant combination therapy (e.g. an opioid antagonist, a GABA B agonist, an NMDA antagonist, a serotonin antagonist, and a cannabinoid antagonist) may be varied and will depend upon the effective dose of each ingredient. Generally, an effective dose of each will be used. Thus, for example, when an opioid antagonist, e.g., naltrexone, is combined with a GABA B agonist, e.g., baclofen, the weight ratio of the opioid antagonist to GABA B agonist will generally range from about 1000:1 to about 1:1000, preferably about 5, 10, 15, 20, 25, 30, 40, 50, 75, 100, 125, 150, 175, 200, 225, 250, 500, 750, 850 and 1,000:1 to about 1:5, 10, 15, 20, 25, 30, 40, 50, 75, 100, 125, 150, 175, 200, 225, 250, 500, 750, 850 and 1,000. Compositions of the agents in the combinations of the present invention (e.g. an opioid antagonist, a GABA B agonist, an NMDA antagonist, a serotonin antagonist, and a cannabinoid antagonist) will generally also be within the aforementioned range, but in each case, an effective dose of each active ingredient should be used.

The active agents employed in the instant combination therapy can be administered in such oral forms as tablets, capsules (each of which includes sustained release or timed release formulations), pills, powders, granules, elixirs, tinctures, suspensions, syrups, and emulsions. The instant invention includes the use of both oral rapid-release and time-controlled release pharmaceutical formulations (see, e.g., U.S. Pat. No. 6,495,166; 5,650,173; 5,654,008 which describes controlled release formulations and is incorporated herein by reference).

The active agents described herein can be administered in a mixture with pharmaceutical diluents, excipients or carriers (collectively referred to herein as “carrier” materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with a non-toxic, pharmaceutically acceptable, inert carrier such as lactose, starch, sucrose, glucose, modified sugars, modified starches, methyl cellulose and its derivatives, dicalcium phosphate, calcium sulfate, mannitol, sorbitol and other reducing and non-reducing sugars, magnesium stearate, steric acid, sodium stearyl fumarate, glyceryl behenate, calcium stearate and the like. For oral administration in liquid form, the drug components can be combined with non-toxic, pharmaceutically acceptable inert carrier such as ethanol, glycerol, water and the like. Moreover, when desired or necessary, suitable binders, lubricants, disintegrating agents and coloring and flavoring agents can also be incorporated into the mixture. Stabilizing agents such as antioxidants (BHA, BHT, propyl gallate, sodium ascorbate, citric acid) can also be added to stabilize the dosage forms. Other suitable components include gelatin, sweeteners, natural and synthetic gums such as acacia, tragacanth or alginates, carboxymethylcellulose, polyethylene glycol, waxes and the like. For a discussion of dosing forms, carriers, additives, pharmacodynamics, etc., see Kirk-Othmer Encyclopedia of Chemical Technology, Fourth Edition, 1996, 18:480-590, incorporated herein by reference. The patient is preferably a mammal, with human patients especially preferred.

This invention is further illustrated by the following examples which should not be construed as limiting. The contents of all the references, patents and published patent applications cited throughout are incorporated herein by reference.

### EXAMPLES

Experimental Procedures

A. General Methods

Animals

Male Wistar rats (initial weight of 200±30 grams; Charles River Laboratories, MA) were individually housed with free access to food and water. The vivarium was maintained on a 12 hour light/dark cycle with a room temperature of 22±3° C.

Drug Preparation

Naltrexone (0.05-10 mg/mL) was prepared daily in 0.9% saline and administered subcutaneously (SC). The drugs AM-251 (03-3.0 mg/mL) and baclofen (0.3-3.0 mg/mL) were suspended in 3% carboxymethyl cellulose; a total volume of 1 mL/kg of this suspension was delivered orally (PO) to the rat using a gavage tube. The two novel opioid antagonist compounds RDC-0313-01 (ALK-101) and RDC-5815-01 (ALK-102) were prepared in 0.9% saline for SC injections (0.0008-0.1 mg/mL) and in 3% carboxymethyl cellulose for oral (PO) administration (at a concentration of 10mg/mL) via gavage. Source of the test compounds are provided in Table 1.

TABLE 1
Drug Information

| COMPOUND | SOURCE |
|---|---|
| Naltrexone | Sigma, Inc. |
| AM-251 | Tocris |
| R(+)-Baclofen | Sigma-Aldrich |
| RDC-0313-01 | Rensselaer Polytechnic Institute |
| RDC-5815-01 | Rensselaer Polytechnic Institute |

Hotplate Test as Measure of Analyesia

The hotplate test is a measure of an animal's response to painful stimuli. Animals are placed on a heated (52° C.) surface confined by a clear, acrylic cage for a maximum of 60 seconds. The animal's behavior on the hotplate is monitored, and the latency to respond, defined as the time for the animal to lick a hind paw in response to the heat, is recorded. When the opioid agonist and analgesic, morphine (15 mg/kg, IP) is administered to an animal 30 minutes prior to hotplate testing, the time when the animal response approaches the maximum allowable latency (60 seconds). In contrast, non-treated or vehicle treated rats will typically respond to the heat with 20 seconds.

Ethanol Self Administration Training Procedure

Animals were trained daily in an operant chamber to press a lever to receive access to an ethanol cocktail as a reinforcer using a saccharin fading procedure. This procedure began with a highly sweetened saccharin solution (0.1%) and increasing amounts of ethanol were gradually introduced over a period of 2-3 weeks while the saccharin was continually reduced. The final ethanol cocktail contained 10% ethanol in 0.04% saccharine. Each session lasted 30 minutes, during which the rat could press the lever twice to gain access to 0.1 mL of the ethanol cocktail. The operant chamber (Coulboume Instruments, Allentown, Pa.) is a computer-controlled automated system which recorded the number of lever presses completed by a rat. At the end of the training period (6-8 weeks), rats which consistently drank a minimum intake of 0.6 g/kg/hour of EtOH (approximately 60 bar presses) were selected to participate in the drug studies. These trained rats were used repeatedly throughout these studies to control for intra-subject variability. Assessments of drug effects were made following a single dosing, with a minimum of a 2 day drug washout period between arms of studies.

### Example I

### Pharmacokinetic (PK) Profile of Novel Opioid Antagonists

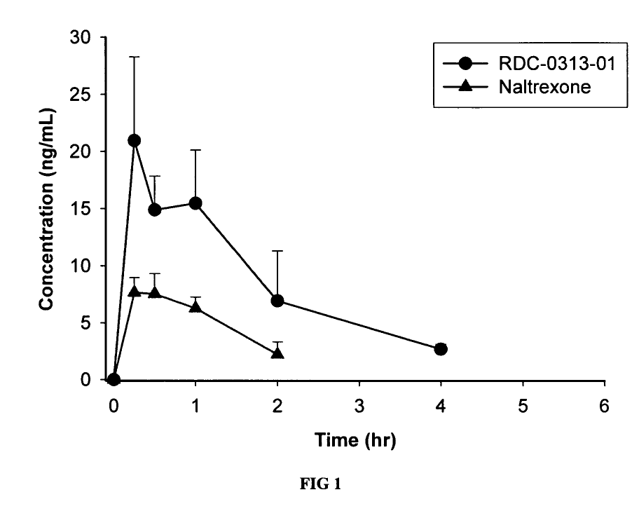

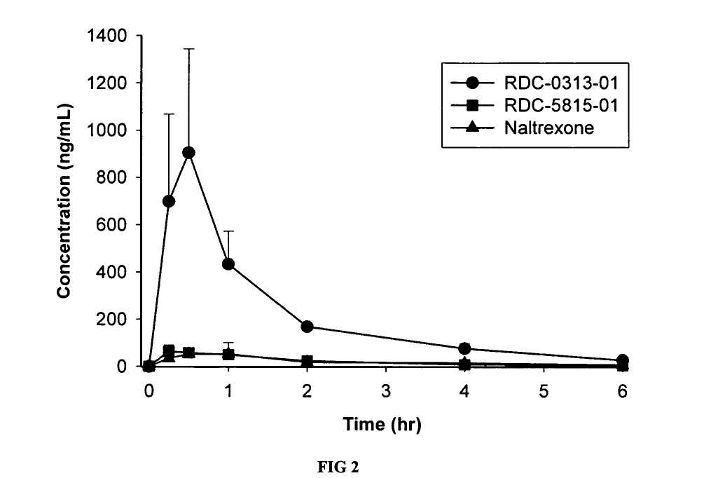

The PK profiles of two novel opioid antagonist compounds, having the same pentacyclic nucleus as naltrexone, were assessed. These studies were designed to directly evaluate the pharmacokinetics of RDC-0313-01 and RDC-5815-01 against naltrexone following intravenous (IV), oral (PO) and subcutaneous (SC) administration (note: RDC-5815-01 was not evaluated by SC route of administration). Male Sprague Dawley rats (n=4 per route of administration per compound) received single IV (1 mg/kg), PO (10 mg/kg), or SC (0.1 mg/kg) doses. Blood samples were collected for 6 hours post-dose. Concentrations of each parent drug were determined by LC/MS-MS. Pharmacokinetic parameters were determined by noncompartmental analysis. RDC-0313-01, RDC-5815-01 and naltrexone were all rapidly absorbed and had similar half-life values. Compared to naltrexone, RDC-03130-01 exposure (AUC) was approximately 8 fold greater following PO administration (FIG. 1) and nearly 2 fold greater following SC administration (FIG. 2). It should be noted that the PK differences may be partially due to absorption and/or metabolic processes. RDC-5815-01 PK was similar to naltrexone. The oral bioavailabilities of RDC-0313-01, RDC-5815-01 and naltrexone were 15%, 6% and 3%, respectively (Table 2).

TABLE 2
Pharmacokinetic Parameters

| | |
|---|---|
| PO (10 mg/kg) | SC (0.1 mg/kg) |

| | | | |
|---|---|---|---|
| AUC∞ | | AUC ∞ | |
| (ng-hr/mL) | F % | (ng-hr/mL) | F % |

| | | | | | |
|---|---|---|---|---|---|
| RDC-0313-01 | 1316 | (357) | 15 | 38.9 (7) | 44 |
| RDC-5818-01 | 149 | (51) | 6 | nd | nd |
| Naltrexone | 150 | (114) | 3 | 14.4 (5) | 31 |

### Example II

### Inhibition of Morphine-Induced Analgesia

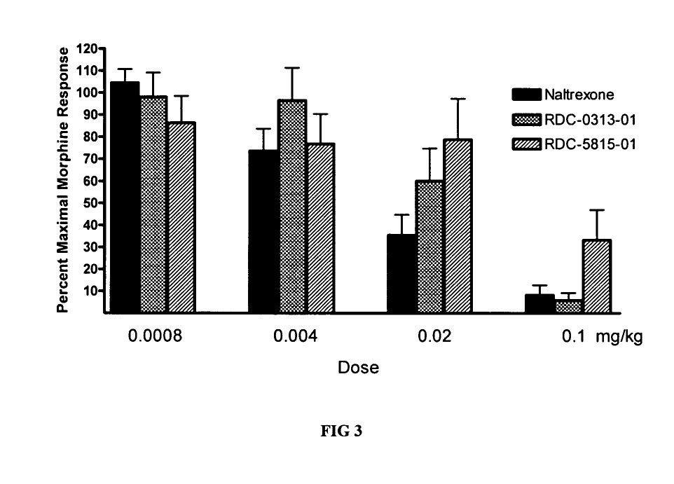

The ability of the opioid antagonists RDC-0313-01, RDC-5815-01 and naltrexone to inhibit morphine-induced analgesia was directly compared on the hotplate test. The antagonists (0.0008-0.1 mg/kg, SC) were administered 30 minutes prior to morphine administration (15 mg/kg, IP) in different groups of rats. Thirty minutes later, the animals were tested on the hotplate. Compared to naltrexone (FIG. 3), RDC-0313-01 was equipotent or slightly less potent (similar dose-response effect), whereas RDC-5815-01 was less potent.

### Example III

### Duration of Action of Opioid Antagonists in Blocking Morphine-Induced Analgesia

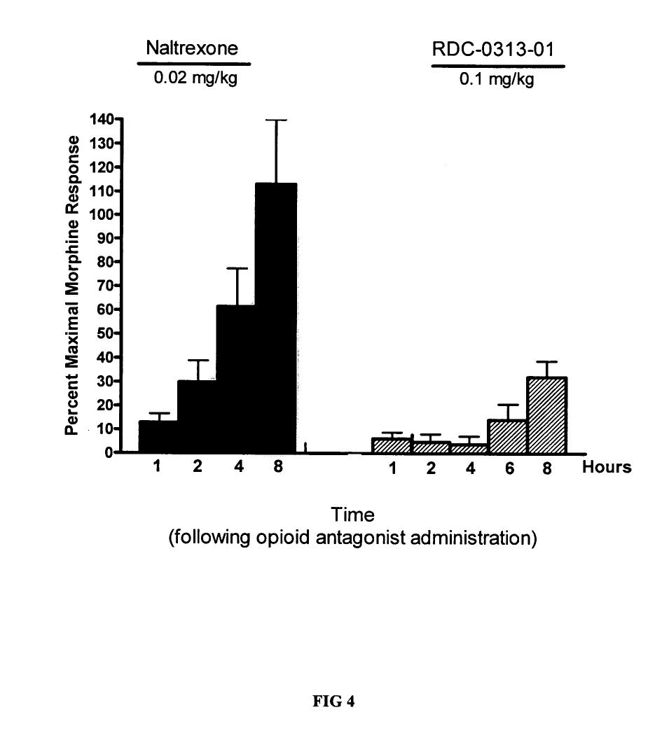

The duration of the blocking effects of naltrexone or RDC-0313-01 on morphine-induced analgesia was determined by testing different groups of animals on the hotplate test from 1 to 8 hours following opioid antagonist administration. Animals were dosed with the opioid antagonists (0.02 or 0.1 mg/kg, SC to approximate equivalent opioid blockade at Hour 1) and 30 minutes prior to hot plate testing, the animals were challenged with morphine (15 mg/kg, IP). The ability of the opioid antagonists to block morphine-induced analgesia decreased with time following treatment, with RDC-0313-01 having a longer duration of action compared to naltrexone (FIG. 4).

### Example IV

### Determination of Tolerance Following Repeated Dosing of Opioid Antagonists on Morphine-Induced Analgesia

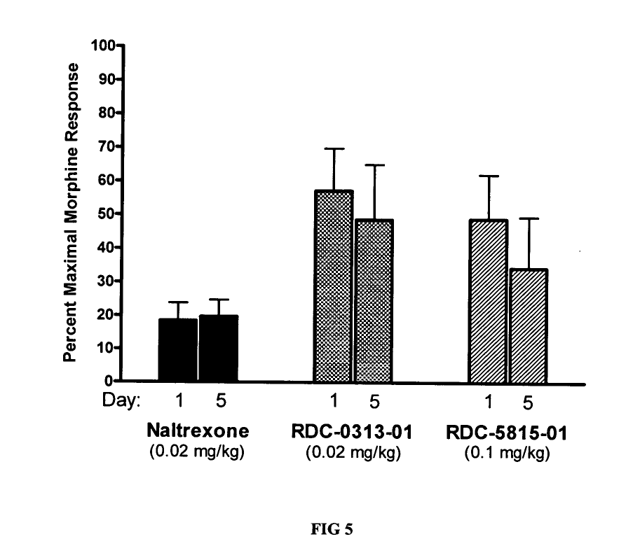

To evaluate the effects of repeated daily dosing on the development of tolerance, the opioid antagonists naltrexone (0.2 mg/kg), RDC-313-01 (0.02 mg/kg) and RDC-5818-01 (0.1 mg/kg, SC) were administered for 5 consecutive days. The dose of RDC-5815-01 was adjusted higher to produce an equivalent pharmacodynamic effect with the other opioid antagonists tested. All animals were dosed at approximately the same time each day. On the first and last day of opioid antagonist treatment, animals were challenged with morphine (15 mg/kg, IP) 30 minutes prior to the hot plate test. No differences in response latencies were observed between the first and last day of treatment suggesting no development of tolerance in the ability of these antagonists to block morphine-induced analgesia (FIG. 5).

### Example V

### Effect of Naltrexone on Ethanol Drinking

The ability of naltrexone to reduce ethanol drinking (i.e., decrease the number of lever presses) was assessed in this animal model of self administration of ethanol. Thirty minutes after the administration of naltrexone (0-6 mg/kg, SC), the animals were placed in the operant chamber and allowed to lever press for the 10% ethanol cocktail. The total number of lever presses was recorded over the 30 minute test session. The rats were repeatedly dosed with naltrexone to generate a dose-response curve for each individual animal. To determine if naltrexone specifically decreased ethanol drinking (as opposed to drinking in general), a 0.1% saccharine solution was substituted for the ethanol cocktail.

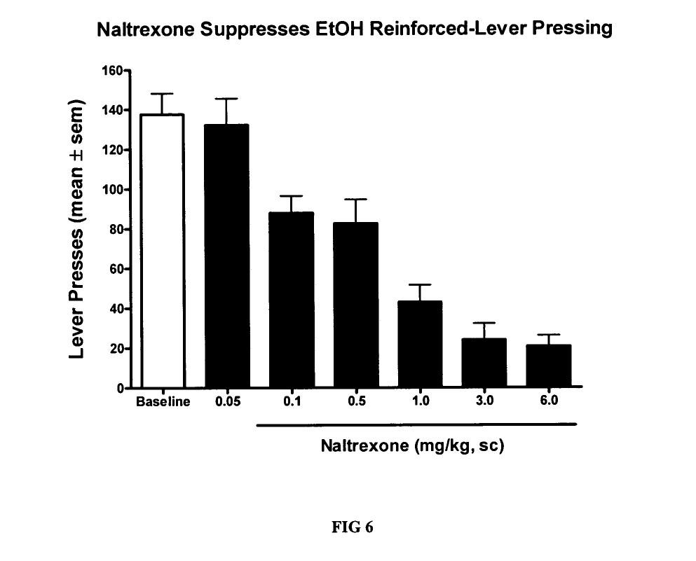

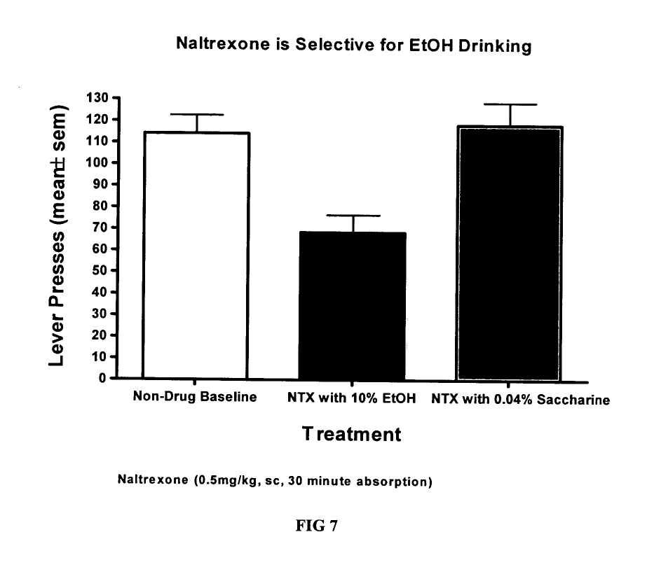

Efficacy of naltrexone was confirmed in the behavioral model of ethanol self administration, as indicated by a dose-dependent decrease in the number of lever presses by treated rats (Table 3, FIG. 6). In contrast, there was no significant decrease between the baseline (no drug treatment), vehicle control (saline) and the lowest dose of naltrexone tested (0.05 mg/kg). At the higher doses (3 and 6 mg/kg), the effect of naltrexone on decreasing ethanol drinking appeared to plateau (bottom out). Additionally, naltrexone was shown at this dose to be selective for decreasing ethanol drinking (self-administration) in rats, but not saccharine drinking (FIG. 7).

TABLE 3
Naltrexone Dose-Response

| | | | | Approximate |
|---|---|---|---|---|
| | Dose | | Lever Presses | Absolute Ethanol |
| Treatment | (mg/kg) | N | (Mean ± SEM) | Consumed (g/kg) |
| No Drug | — | 9 | 138 ± 10.6 | 1.1 |
| (Baseline) | | | | |
| Naltrexone | 0.05 | 9 | 132 ± 13.3 | 1.0 |
| | 0.1 | 9 | 88 ± 8.6 | 0.7 |
| | 0.5 | 8 | 83 ± 12.1 | 0.6 |
| | 1.0 | 7 | 43 ± 8.6 | 0.3 |
| | 3.0 | 6 | 24 ± 8.1 | 0.2 |
| | 6.0 | 6 | 21 ± 5.5 | 0.2 |

### Example VI

### Effect of Novel Opioid Antagonists on Ethanol Drinking

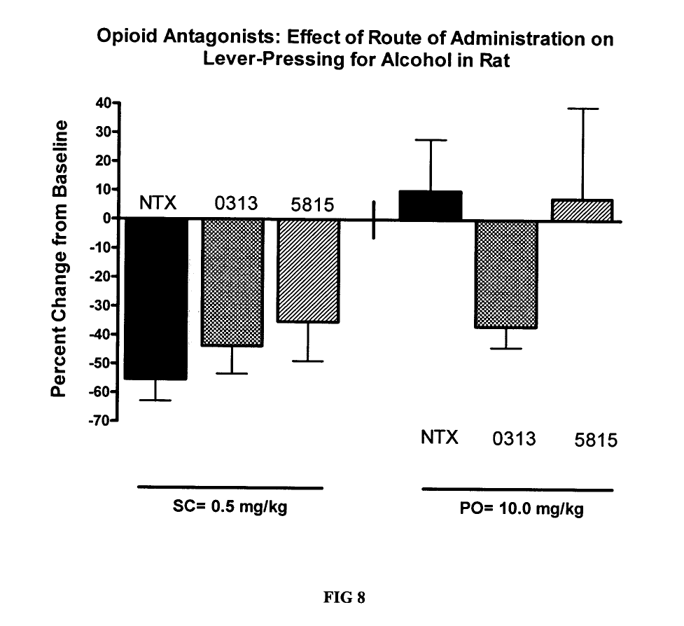

To study the effects of the novel opioid antagonists RDC-0313-01 and RDC-5818-01 on the self-administration of ethanol, the compounds were administered (0.5 mg/kg, SC) 30 minutes prior to testing in the operant chambers and directly compared with naltrexone. In addition, oral activity was also assessed. The animals were dosed by oral gavage with a 10 mg/kg solution of naltrexone or the RDC compounds. The animals were tested in the operant chambers one hour later and the number of lever presses for ethanol was recorded for the 30 minute session. When administered SC, the opioid antagonists had equivalent effects on decreasing the self-administration of ethanol. However, when administered orally, only RDC-0313-01 was active in decreasing ethanol self-administration (FIG. 8).

### Example VII

### Effect of the Co-Administration of Other Drugs with Naltrexone on Ethanol Drinking

The CB1 antagonist (AM-251) and the GABA B agonist (baclofen) were coadministered with naltrexone to determine if it affected naltrexone's ability to decease ethanol drinking. The dose of naltrexone used in this series of studies was the ED₇₅ (that is, the dose of naltrexone that produced a 25% decrease in lever responses for ethanol as determined from the dose-response study). This dose allows one to determine if the co-administered drugs impaired or enhanced naltrexone's effect on ethanol drinking. The drugs were administered orally 30 minutes prior to a naltrexone injection (SC), and 60 minutes prior to the beginning of the ethanol drinking test session. The number of lever presses for the ethanol cocktail was recorded at the end of the 30 minute session.

### Example VIII

### Effect of the Coadministration of Cannabinoid CB₁ Antagonist with Naltrexone on Ethanol Drinking

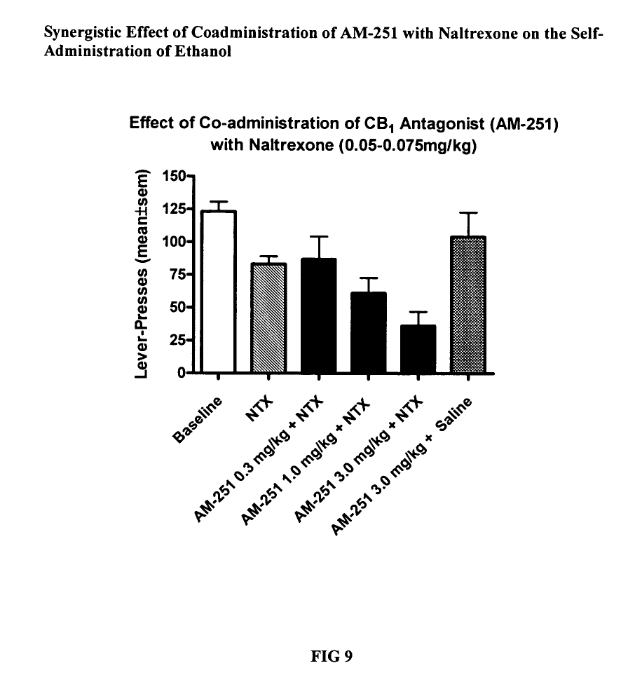

This phase of the study investigated the effect of potential drug interactions between naltrexone and a cannabinoid CB₁ antagonist (AM-251) on the number of lever presses by rats for ethanol compared to naltrexone alone. A significantly higher number of lever responses would demonstrate that the drug interaction impaired naltrexone's ability to decrease ethanol drinking. In contrast, significantly lower responses would suggest a synergistic or additive effect of the drug combination.
Acute Dosine. A range of doses of AM-251 (0.3-3.0 mg/kg) were administered orally together with a low dose of naltrexone (SC; ED₇₅, 0.05-0.075 mg/kg; titrated for each individual animal) to examine the potential drug interaction on ethanol drinking. Naltrexone decreased the number of lever presses for ethanol by 32.5% compared to non-drug treated (baseline) conditions. A further significant decrease in ethanol drinking was observed with AM-251 (1.0 or 3.0 mg/kg) plus naltrexone (0.05-0.075 mg/kg) compared to naltrexone alone (p<0.05 and 0.001, respectively). This attenuation in ethanol drinking was not seen when the lower dose of AM-251 (0.3 mg/kg) was coadministered with naltrexone. Further, AM-251 at 3.0 mg/kg alone had no effect on lever pressing for ethanol in this model compared to non-drug treated conditions (FIG. 9).

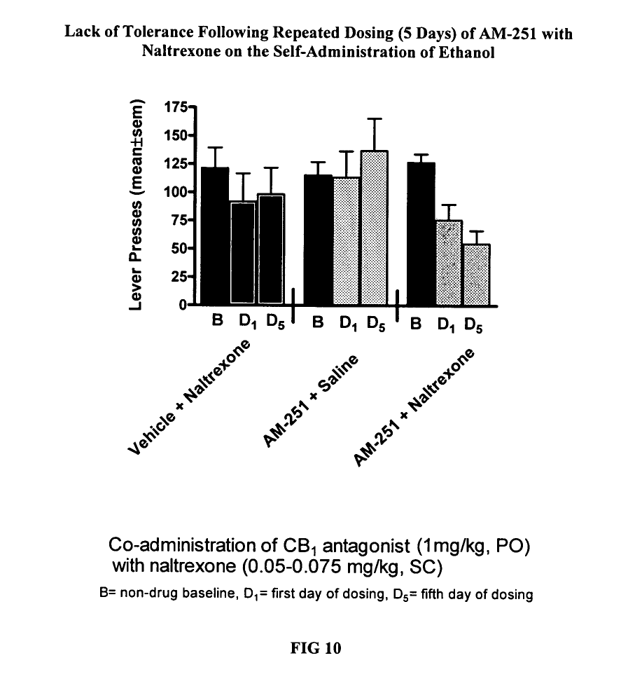

Repeated Dosing. The previous studies were conducted using a single dosing procedure. To determine if this drug interaction might be further enhanced with repeated daily dosing or conversely, the effect on decreasing alcohol self-administration is lost (due to tolerance) following multiple daily dosing, the study was repeated with a once-a-day for 5 days dosing procedure. The dose of AM-251 was 1 mg/kg (sub-maximal dose to allow for the observation of a potentiation or attenuation of the acute dose effect) together with the ED₇₅ of naltrexone. Animals were tested on Day 1 and on Day 5. As seen with the initial single exposure dosing study, the coadministration of AM-251 with naltrexone decreased the number of lever presses for ethanol compared with naltrexone or AM-251 alone (FIG. 10). No differences in the number of lever presses were observed between the first day of dosing and the last, suggesting a lack of tolerance over time (Table 4).

TABLE 4
Percent Change from Non-Drug Baseline: Comparison
of Single versus Repeated Dosing

| | | Repeated Dosing: | Repeated Dosing: |
|---|---|---|---|
| Treatment | Single Dosing | Day 1 | Day 5 |
| AM-251 + Saline | N/A | −2% | +19% |
| Vehicle + NTX | −33% | −24% | −19% |
| AM-251 + NTX | −50% | −40% | −57% |

### Example IX

### Effect of the Coadministration of Baclofen (GABA_{B} Agonist) with Naltrexone on Ethanol Drinking

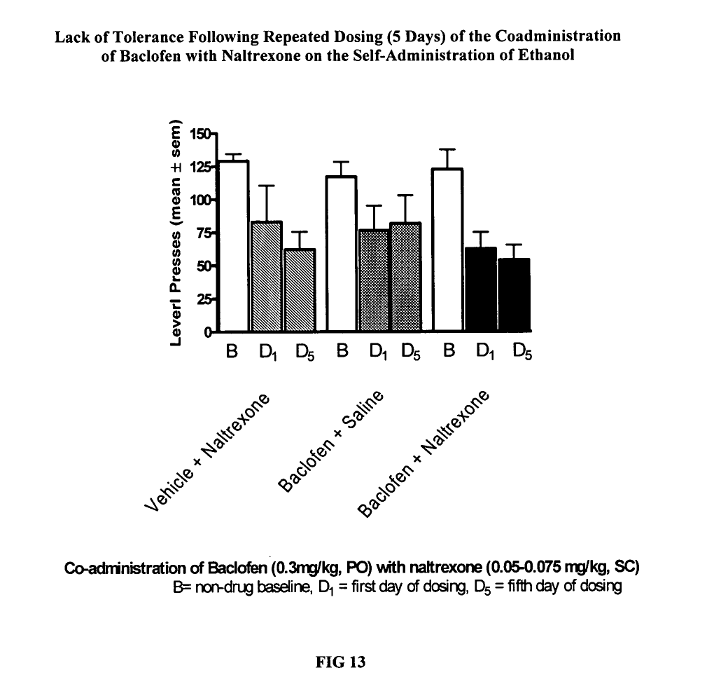

This phase of the study investigated the effect of potential drug interactions between naltrexone and a GABA_{B} agonist (baclofen) on the number of lever presses by rats for ethanol compared to naltrexone alone. A significantly higher number of lever responses would demonstrate that the drug interaction impaired naltrexone's ability to decrease ethanol drinking. In contrast, significantly lower responses would suggest a synergistic or additive effect of the drug combination.
Single Dosing. A range of doses of baclofen (0.3-3.0 mg/kg) were administered orally together with a low dose of naltrexone (SC; ED₇₅, 0.05-0.075 mg/kg titrated for each individual animal) to examine the potential drug interaction on ethanol drinking. Naltrexone decreased the number of lever presses for ethanol by 18% compared to non-drug treated (baseline) conditions. A further significant decrease in ethanol drinking was observed with baclofen (1.0 or 3.0 mg/kg) plus naltrexone (0.05-0.075 mg/kg) compared to naltrexone alone (p<0.01). Further, baclofen at 3.0 mg/kg alone had a significant effect on lever pressing for ethanol in this model compared to non-drug treated conditions (p<0.01, FIG. 13), suggesting that the effects of the coadministration of baclofen with naltrexone were an additive effect. A dose response with baclofen alone was run and again only the high dose (3.0 mg/kg) significantly decreased the self-administration of ethanol (p<0.01, FIG. 14).

Repeated Dosing. The previous study was conducted using a single dosing procedure. To determine if this drug interaction might be further enhanced with repeated dosing or conversely, the effect on decreasing alcohol self-administration is lost (due to tolerance) following multiple daily dosing, the study was repeated with a once-a-day for 5 days dosing procedure. The dose of baclofen was 0.3 mg/kg (sub-maximal dose to allow for the observation of a potentiation or attenuation of the acute dose effect) together with the ED₇₅ of naltrexone. Animals were tested on Day 1 and on Day 5. As seen with the initial single dosing study, the coadministration of baclofen with naltrexone decreased the number of lever presses for ethanol compared with naltrexone or baclofen alone (FIG. 13). No differences in the number of lever presses were observed between the first day of dosing and the last, suggesting a lack of tolerance over time (Table 5).

TABLE 5
Percent Change from Non-Drug Baseline: Comparison
of Acute versus Sub-chronic Dosing

| | | Repeated Dosing: | Repeated Dosing: |
|---|---|---|---|
| Treatment | Single Dosing | Day 1 | Day 5 |
| Baclofen + Saline | N/A | −35% | −30% |
| Vehicle + NTX | −18% | −43% | −57% |
| Baclofen + NTX | −46% | −41% | −56% |

### REFERENCES

Gold, M. S. and J. Star. Eating Disorders. In: J. H. Lowenson, P. Ruiz, R. B. Millman and J. G. Langrod (eds.), Substance Abuse: A Comprehensive Textbook, Lippincott Williams and Wilkins, 2005, pp 469-488.
O'Leary G, Borrow J and Weiss R D. Opioid antagonists in the treatment of alcohol dependence. Current Psychiatry Report, 2001 December;3(6):484-488.
Oswald L M and Wand G S. Opioids and alcoholism. Physiolology and Behavior, 2004 April;81(2):339-358.
Modesto-Lowe V and Van Kirk J. Clinical uses of naltrexone: a review of the evidence. Experimental and Clinical Psychopharmacology, 2002 August; 10(3):213-227.
Mann K. Pharmacotherapy of alcohol dependence: A review of the clinical data. CNS Drugs. 2004; 18(8):485-504.
Vetulani J. Drug addiction. Part III. Pharmacotherapy of addiction. Polish Journal of Pharmacology. 2001 September-October;53(5):415-434.
Gentry J R, Hill C and Malcolm R. New anticonvulsants: A review of applications for the management of substance abuse disorders. Annuals of Clinical Psychiatry. 2002 December;14(4):233-245.
Cornish, J W, McNicholas and C P O'Brien. Treatment of Substance-Related Disorders. In: A F Schatzberg and C B Nemeroff (eds.), Textbook of Psychopharmacology, American Psychiatric Publishing, 2004, pp 1009-1030.
Kim S W, Grant J E and Grosz R L. Pathological gambling. Current status and new treatments. Minnesotta Medicine 2002 July;85(7):48-52.
Agras, W S. Treatment of Eating Disorders. In: A F Schatzberg and C B Nemeroff (eds.), Textbook of Psychopharmacology, American Psychiatric Publishing, 2004, pp 1031-1040.
Henningfield J E, Fant R V, Buchhalter A R and Stitzer M L. Pharmacotherapy for nicotine dependence. CA: A Cancer Journal for Clinicians, 2005 September-October;55(5):281-299.

What is claimed is:

1. A pharmaceutical composition for the treatment of brain reward system disorders comprising concurrently administering to a subject in need of treatment a therapeutically effective amount of: (i) a first compound comprising an opioid antagonist or a pharmaceutically acceptable salt, isomer, prodrug, analog, metabolite or derivative thereof; and (ii) and a second compound effective to ameliorate or eliminate at least one symptom of brain reward system disorders; wherein the combined therapy potentiates the therapeutic response compared to treatment of either compound as monotherapy.

2. The composition of claim 1, wherein said antagonist is mu receptor-selective.

3. The composition of claim 2, wherein said antagonist is selected from the group consisting of naltrexone, naloxone, and nalmefene or a pharmaceutically acceptable salt, isomer, prodrug, analog, metabolite or derivative thereof.

4. The composition of claim 3, wherein said antagonist is naltrexone.

5. The composition of claim 1, wherein said antagonist is delta receptor-selective.

6. The composition of claim 1, wherein said antagonist is kappa receptor-selective.

7. The composition of claim 1, wherein said opioid antagonist of the present invention is represented by the structure of the following formula: 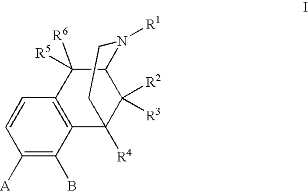
R¹ is selected from the group consisting of hydrogen, a substituted or unsubstituted, saturated or unsaturated aliphatic group, a substituted or unsubstituted, saturated or unsaturated alicyclic group, a substituted or unsubstituted aromatic group, a substituted or unsubstituted heteroaromatic group, or saturated or unsaturated heterocyclic group;
R² is selected from the group consisting of hydrogen, hydroxy, alkoxy, amino or substituted amino;
R³ and R⁴ are aliphatic;
R³ and R⁴ are taking together to form the following formula II: 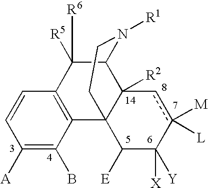
R⁵ and R⁶ are both hydrogen or taken together R⁵ and R⁶ are ═O;
A, B and E are independently selected from hydrogen, halogen, R¹, OR¹, SR¹, CONR³R⁴ and NR³R⁴; wherein R³ and R⁴is independently selected from the group consisting of hydrogen, acyl, a substituted or unsubstituted, saturated or unsaturated aliphatic group, a substituted or unsubstituted, saturated or unsaturated alicyclic group, a substituted or unsubstituted aromatic group, a substituted or unsubstituted heteroaromatic group, saturated or unsaturated heterocyclic group; or can be taken together with the nitrogen atom to which they are attached to form a substituted or unsubstituted heterocyclic or heteroaromatic ring;
B and E are taken together to form the following formula III: 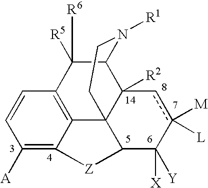
wherein Z is selected from O,S, or NR¹;
X and Y are independently selected from the group consisting of hydrogen, deuterium, halogen, nitrile, azide, R₁, OR₁, S(O)ₙR¹, —NR¹C(O)R¹, —NR¹C(O)NR³R⁴, —NR¹S(O)ₙR¹, —CONR³R⁴, and NR³R⁴;
or X and Y, taken together with the carbon atom to which they are attached, are selected from the group consisting of CO, C═CHR¹, C═NR¹, C═NOR¹, C═NO(CH₂)ₘR¹, C═NNHR¹, C═NNHCOR¹, C═NNHCONR¹R², C═NNHS(O)ₙR¹, or C═N—N═CHR¹;
R² and either X or Y taken together to form an additional sixth ring, which may be saturated or unsaturated;
L and M are independently selected from the group consisting of hydrogen, R₁, OR₁;
or L and M, taken together with the carbon atom to which they are attached, is selected from the group consisting of C═CHR¹, or a C₃-C₁₀ spiro-fused carbocycle;
L and Y can be taken together to form a fused substituted or unsubstituted aryl or heteroaryl.

8. The composition of claim 1, wherein said second compound is selected from the group consisting of a GABA B agonist, an NMDA antagonist, a serotonin antagonist, and a cannabinoid antagonist.

9. The composition of claim 8, wherein said GABA B agonist is baclofen.

10. The composition of claim 8, wherein said NMDA antagonist is memantine.

11. The composition of claim 8, wherein said serotonin antagonist is selected from the group consisting of buspirone, ondansetron and granisetron.

12. The composition of claim 8, wherein said cannabinoid antagonist is SR-141716A or AM-251.

13. The composition according to claim 1, wherein the opioid antagonist in said composition is administered in a daily dose ranging from about from about 1 mg to about 500 mg and the second compound in said composition is administered in a daily dose ranging from about from about 1 mg to about 500 mg.

14. The composition of claim 1, wherein said composition further comprises a sustained release carrier such that the dosage form is administrable on a twice-a-day or on a once-a-day basis.

15. The composition of claim 14, wherein the sustained release carrier causes said composition to be released over a time period of about 8 to about 24 hours when orally administered to a human patient.

16. The composition of claim 15, wherein said sustained release carrier is formulated as a tablet, capsule, pill, lozenge or potion.

17. The composition of claim 1, wherein the symptoms ameliorated or eliminated include anxiety, nausea, excitability, insomnia, craving, irritability, impulsivity, anger or rage.

18. The composition of claim 1, wherein the therapeutic response is a synergistic or additive effect.

19. The composition of claim 1, wherein the brain reward system disorder is selected from the group comprising pathological gambling, compulsive alcohol consumption, compulsive over-eating and obesity, compulsive smoking, and drug addiction.

20. A composition comprising a combination of an opioid antagonist and a second compound that effectively ameliorates or eliminates at least one symptom of a brain reward system disorder in a pharmaceutically acceptable carrier.

21. Use of a composition comprising the combination according to any one of claims 1 to 19, for the treatment of a brain reward system disorder.

22. A method for the treatment of brain reward system disorders comprising concurrently administering to a subject in need of treatment a therapeutically effective amount of: (i) a first compound comprising an opioid antagonist or a pharmaceutically acceptable salt, isomer, prodrug, analog, metabolite or derivative thereof; and (ii) and a second compound effective to ameliorate or eliminate at least one symptom of an brain reward system disorder.

23. A method for changing brain reward system disorders behavior of a subject suffering from withdrawal symptoms associated with alcohol abuse comprising administering a therapeutically effective amount of: (i) a first compound comprising an opioid antagonist or a pharmaceutically acceptable salt, isomer, prodrug, analog, metabolite or derivative thereof; and (ii) and a second compound effective to ameliorate or eliminate at least one symptom of a brain reward system disorder.

24. An orally administrable dosage form containing the pharmaceutical composition of claim 1, wherein said dosage form provides a once daily dosing for therapeutic relief of at least one symptom of a brain reward system disorder.

25. A sustained-release formulation for the treatment of brain reward system disorders comprising concurrently administering to a subject in need of treatment a therapeutically effective amount of: (i) a first compound comprising an opioid antagonist or a pharmaceutically acceptable salt, isomer, prodrug, analog, metabolite or derivative thereof; and (ii) and a second compound effective to ameliorate or eliminate at least one symptom of brain reward system disorders; wherein the combined therapy potentiates the therapeutic response compared to treatment of either compound as monotherapy.

26. A pharmaceutical kit comprising an oral dosage form of a first compound comprising an opioid antagonist and a second compound that effectively ameliorates or eliminates at least one symptom of a brain reward system disorder.
